(19) United States
(12) Patent Application Publication
(10) Pub. No.: US 2017/0325700 A1
(43) Pub. Date: Nov. 16, 2017

(54) REAL-TIME VAGAL MONITORING AND INTERVENTION

(71) Applicant: Arizona Board of Regents on Behalf of the University of Arizona, Tucson, AZ (US)

(72) Inventors: Richard LANE, Tucson, AZ (US); Janet Meiling WANG, Tucson, AZ (US); John ALLEN, Tucson, AZ (US)

(21) Appl. No.: 15/519,771

(22) PCT Filed: Oct. 16, 2015
(86) PCT No.: PCT/US15/56034

§ 371 Date: Apr. 17, 2017

Related U.S. Application Data
(60) Provisional application No. 62/064,943, filed on Oct. 16, 2014.

(51) Int. Cl. A61B 5/0402; A61B 5/024; A61B 5/00; A61B 5/0245; G06F 19/00

(52) U.S. Cl. CPC A61B 5/0402; A61B 5/742; A61B 5/0245; A61B 5/02405; G06F 19/3418; G06F 19/3431

(57) ABSTRACT

Real-time vagal monitoring and intervention is provided. An exemplary system for real-time vagal monitoring and intervention may include a monitor device. Such a monitor device may include one or more electrocardiograph (ECG) electrodes that detects electrical activity in a heart of the person, a processor that executes instructions to calculate multiple measures of heart rate variability (HRV) based on electrical activity detected by the ECG electrodes, and a wireless interface that continuously transmits each calculated HRV measure over a wireless network to a user device.

### BACKGROUND OF THE INVENTION

### 1. Field of Technology

The present invention generally relates to vagal tone. More specifically, the present invention relates to monitoring and intervening to increase cardiac vagal tone in real-time.

### 2. Description of the Related Art

The human body is capable of responding to challenges and threats with an orchestrated set of physiological responses that facilitate adaptive behaviors. Such responses may be part of a “fight or flight” response and are mediated by a system that includes a network of nerves connecting the brain and body called the sympathetic nervous system. When the sympathetic nervous system is stimulated, a number of bodily changes may occur. In that regard, an over-expressed sympathetic response may manifest as hypertension, elevated heart rate, elevated cardiac output, and a hyper-inflammatory state. Such states may contribute to the symptoms of heart disease, disease progression, infarction, arrhythmia, or even heart failure and death. Drugs (e.g., beta blockers, calcium channel blockers, anti-arrhythmic) may be used to limit the effects of an elevated sympathetic response, but may produce side effects such as fatigue, dizziness, bradycardia, and impotence.

A complementary physiological system called the parasympathetic nervous system generally opposes the sympathetic nervous system. This system has the function of rest and digest. The latter functions, which may include slowing down the heart rate, contribute to restoring and conserving energy. The part of the parasympathetic nervous system that slows the heart rate is the vagus nerve. Activating the vagus nerve generally promotes a relaxation response. Vagal tone refers to the general level of activity of the vagus nerve, is a function of respiratory sinus arrhythmia (RSA) and is most commonly measured by heart rate variability (HRV).

Low vagal tone is associated with such conditions as depression, anxiety, stress, and ventricular tachycardia/ventricular fibrillation in vulnerable persons. By contrast, improving vagal tone may provide greater protection against lethal ventricular arrhythmias in vulnerable persons. Lower vagal tone may also be associated with pro-inflammatory effects, including depression symptoms, oxidative stress markers, thrombo-embolic episodes, myocardial and other end-organ ischemia, renal impairment, graft patency, pain, new onset post-operative atrial fibrillation, infection, and impaired wound healing. Reductions in each of the foregoing by increasing vagal tone may potentially contribute to improved person outcomes.

There are presently a variety of ways for doctors and other health care providers to evaluate and provide treatments affecting sympathetic-parasympathetic balance. Such evaluations generally take place in a clinical setting, however, and as noted above, such treatments may involve prescribed drugs, such as sympatholytic medications. Another complication is that vagal tone may differ from person to person, such that one level for one person may be associated with normal health, but the same level may be indicative of a health risk in someone else. For example, vagal tone is on average lower in older individuals. A given level of vagal tone may be healthy in an older person but abnormally low in a younger person.

There is, therefore, a need in the art for improved systems and methods for real-time vagal monitoring and intervention.

### SUMMARY OF THE CLAIMED INVENTION

Embodiments of the present invention allow for real-time vagal monitoring and intervention to increase vagal tone. An exemplary system for real-time vagal monitoring and intervention may include a monitor device. Such a monitor device may include one or more electrocardiograph (ECG) electrodes that detects electrical activity in a heart of the person, a processor that executes instructions to calculate multiple measures of heart rate variability (HRV) based on electrical activity detected by the ECG electrodes, and a wireless interface that continuously transmits each calculated HRV measure over a wireless network to a user device.

Further embodiments may include monitoring devices for real-time vagal monitoring and intervention. Such monitoring device may include one or more sensors that detects electrical activity in a heart of the person, a processor that executes instructions to calculate multiple measures regarding heart rate variability (HRV) based on electrical activity detected by the sensors, and a wireless interface that continuously transmits each calculated HRV measure over a wireless network to a user device that determines when the received HRV measure meets a predetermined HRV risk threshold specific to the person, that identifies a therapeutic intervention associated with the HRV risk threshold, and that provides a visual guide regarding the identified therapeutic intervention.

Additional embodiments may include user devices for real-time vagal monitoring and intervention. Such user devices may include a wireless interface that receives multiple continuous heart rate variability (HRV) measures sent over a wireless communication network from a monitor device where each calculated HRV measures concern electrical activity of a heart of a person, a processor that executes instructions to determine when the received HRV measure meets a predetermined HRV risk threshold specific to the person and to identify a therapeutic intervention associated with the HRV risk threshold, and a display screen that provides a visual guide regarding the identified therapeutic intervention.

Yet further embodiments may include systems for real-time vagal monitoring and intervention. Such systems may include a database in memory that stores information regarding one or more interventions, each intervention associated with a set of one or more triggers, a sensor that detects when one or more triggers has occurred, and a processor that executes instructions to determine that a set of the detected triggers is associated with one of the interventions and to generate instructions for implementing the intervention associated with the detected set of triggers.

Some embodiments may include systems for real-time vagal monitoring and intervention. Such systems may include a database in memory that stores information regarding one or more interventions, each intervention associated with a visual guide, a processor that executes instructions to determine that one of the interventions has been triggered and determines a visual guide associated with the triggered intervention, and a display screen that displays the visual guide determined to be associated with the triggered intervention.

Further embodiments may include systems for real-time vagal monitoring and intervention. Such systems may include a communication interface that receives information sent over a wireless communication network from one or more monitor devices, a processor that executes instructions to determine that the received information from the monitor device indicates a need to launch an identified intervention that uses one or more intervention devices and to generate a set of instructions to each of the intervention devices regarding the identified intervention. The communication interface may then send each set of instructions over the wireless communication network to the associated intervention device.

Yet further embodiments may include systems for real-time vagal monitoring and intervention. Such systems may include a communication interface that receives biodata sent over a wireless communication network from multiple user devices each associated with a different user, memory that stores the received information in a user profile database of user profiles each associated with a respective different user, a processor that executes instructions to identify a user group comprising multiple user based on one or more specifications where the user group comprises multiple users represented in the user profile database and to establish a baseline for the identified user group based on the biodata in the user profiles associated with the users in the user group.

Embodiments may further include methods performed by the foregoing systems, as well as non-transitory computer-readable storage medium having embodied thereon instructions for performing such methods.

### BRIEF DESCRIPTION OF THE DRAWINGS

FIG. 1 illustrates a network environment in which a system for real-time vagal monitoring and intervention may be implemented.

FIG. 2 illustrates a mobile device architecture that may be utilized to implement the various features and processes described herein.

FIG. 3 is a flowchart illustrating a method for real-time vagal monitoring and intervention.

FIG. 4 is diagram of an exemplary system for real-time vagal monitoring and intervention.

### DETAILED DESCRIPTION

Embodiments of the present invention allow for real-time vagal monitoring and intervention to increase vagal tone. An exemplary system for real-time vagal monitoring and intervention may include a monitor device. Such a monitor device may include one or more electrocardiograph (ECG) electrodes that detects electrical activity in a heart of the person, a processor that executes instructions to calculate multiple measures of heart rate variability (HRV) based on electrical activity detected by the ECG electrodes, and a wireless interface that continuously transmits each calculated HRV measure over a wireless network to a user device.

Such a user device may include a wireless interface that receives each calculated HRV measure from the monitor device, and a processor that executes instructions to determine when the received HRV measure meets a predetermined HRV risk threshold specific to the person and to identify a therapeutic intervention associated with the HRV risk threshold. The user device may further include a display screen that provides a visual guide regarding the degree to which vagal tone is changing.

Because vagal tone may be associated with wide-ranging conditions, embodiments of the claimed invention may likewise be customized and applied in a variety of ways. In that regard, specific applications may pertain not only to general health, fitness, meditation, relaxation, yoga, or stress reduction. Such an application may monitor physiological stress level on a momentary, daily, weekly, and/or monthly basis, as well as receive self-reported stress levels and expected physiological adjustments based on exertion data. The disparity between self-reporting and physiology may be tracked with the goal of improving recognition and regulation of stress.

Additional embodiments may pertain to weight loss and specific medical conditions, including heart conditions such as congestive heart failure, atrial fibrillation, implanted defibrillators, symptomatic frequent ventricular arrhythmias (PVCs), bundle branch blocks, and genetically based disorders that may increase risk for sudden death (e.g., Long QT Syndrome, Brugada syndrome). Further medical applications may pertain to hypertension, pulmonary conditions such as asthma or COPD, cancer, diabetes, obesity, epilepsy, inflammatory disorders, etc. Medical conditions may further include psychiatric disorders, including depression, anxiety disorders including PTSD, panic disorder, borderline personality, addictions, first episode or chronic psychosis, somatic symptom disorders, and agitation. Specific medical and psychiatric situations where the application may be used further include pre-surgical, post-surgical, psychiatric therapy, including inpatient treatment and restraints, and psychotherapy.

Non-medical contexts where the application may be used may include various high-stress situations, including artistic and athletic performances or competitions, business presentations and decisions, schools (e.g., test anxiety), and law-enforcement and military contexts.

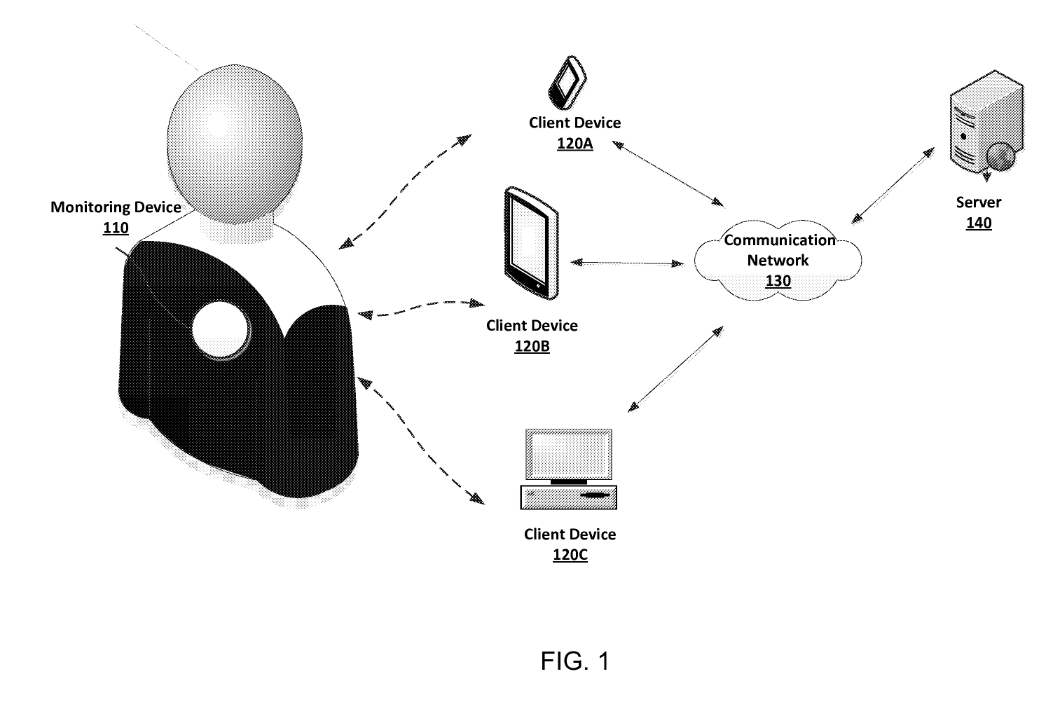

FIG. 1 illustrates a network environment 100 in which a system for real-time vagal monitoring and intervention may be implemented. Network environment 100 may include a monitor device 110, one or more user devices 120A-B, a communication network 140, and a server 150.

Monitor device 110 may include one or more sensors. Such sensors may include electrocardiograph (ECG) sensors that detect and measure electrical activity in the heart of a person when placed in proximity thereto (e.g., against the skin on the chest of the person). Another type of sensor that may be included in monitor device is an actigraph sensor for detecting movement. Such an actigraph sensor may be capable, for example, of detecting and measuring the movement of the chest of a person. Such movements may be indicative of the breathing (e.g., rate, depth) of the person. Any type of medical and health-related biosensors known in the art may also be included in monitor device 110. In this regard, monitor device 110 may be able to gather data regarding respiration, posture, activity level of the person, and other biodata of the person.

In some embodiments, the monitor device 110 may further include an attachment component that holds the sensors in place near the heart. Such an attachment component may include an adhesive, a tape, a strap, a garment, a wearable device, or combinations of the same, as well as other equivalents in the art for attaching a device to a person.

The monitor device 110 may further include a processor that receives the sensor information and calculates multiple measures or metrics based on the sensor information. For example, the processor may calculate an HRV measure based on information regarding the electrical activity of heart as detected by ECG electrodes.

The monitor device 110 may further include a wireless interface for communicating over a wireless network. In some embodiments, the wireless interface may be a Bluetooth interface for communicating over short distances with other Bluetooth-enabled devices (e.g., user devices 120). In exemplary embodiments, the monitor device 110 may send the information gathered by the sensors, as well as the calculations regarding the same, to another device via its wireless interface.

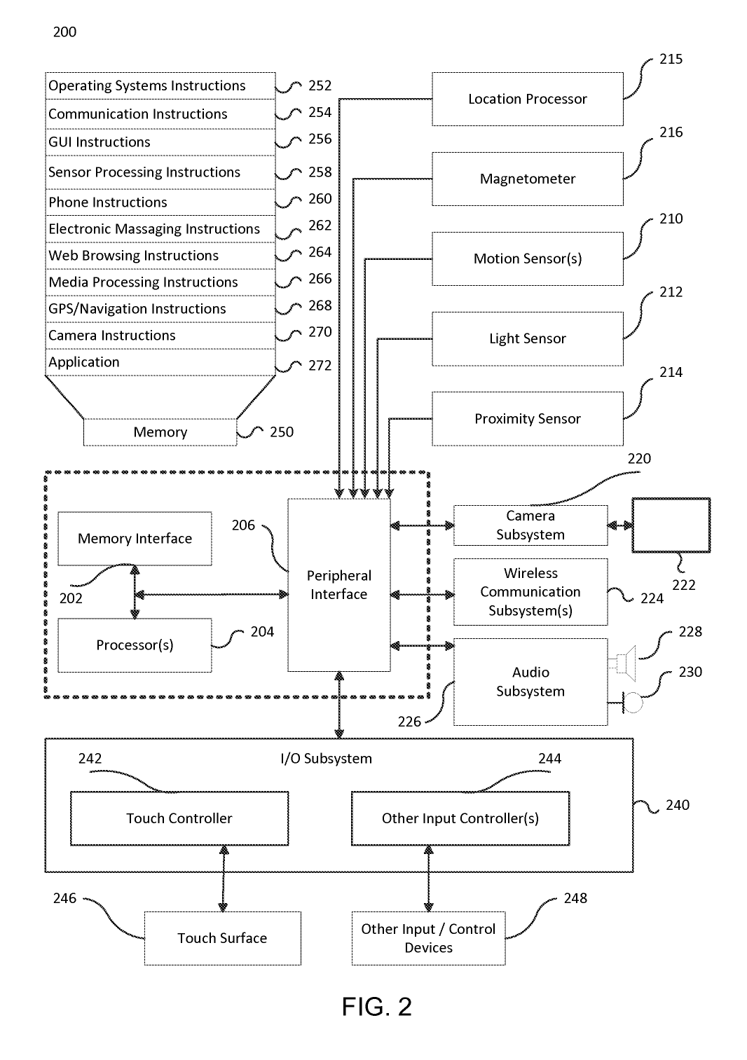

Users may use any number of different electronic user devices 120A-B, such as general purpose computers, mobile phones, smartphones, personal digital assistants (PDAs), portable computing devices (e.g., laptop, netbook, tablets), desktop computing devices, handheld computing device, or any other type of computing device capable of communicating over communication network 130. An exemplary user device is described in further detail with respect to FIG. 2.

As used herein, user device 120 may be used to communicate with monitor device 110, as well as serve as a repository for data specific to the person. For example, such data may include calculated baseline data, threshold data, as well as various health-related parameters regarding stress, anxiety, depression, anger, etc.

Communication network 130 may be a local, proprietary network (e.g., an intranet) and/or may be a part of a larger wide-area network. The communications network 130 may be a local area network (LAN), which may be communicatively coupled to a wide area network (WAN) such as the Internet. The Internet is a broad network of interconnected computers and servers allowing for the transmission and exchange of Internet Protocol (IP) data between users connected through a network service provider. Examples of network service providers are the public switched telephone network, a cable service provider, a provider of digital subscriber line (DSL) services, or a satellite service provider. Communications network 130 allows for communication between the various components of network environment 100.

Server 130 may include any type of server or other computing device as is known in the art, including standard hardware computing components such as network and media interfaces, non-transitory computer-readable storage (memory), and processors for executing instructions or accessing information that may be stored in memory. The functionalities of multiple servers may be integrated into a single server. Any of the aforementioned servers (or an integrated server) may take on certain client-side, cache, or proxy server characteristics. These characteristics may depend on the particular network placement of the server or certain configurations of the server.

FIG. 2 illustrates a mobile device architecture 200 that may be utilized to implement the various features and processes described herein. Architecture 200 can be implemented in any number of portable devices including but not limited to smart phones, electronic tablets, and gaming devices. Architecture 200 as illustrated in FIG. 2 includes memory interface 202, processors 204, and peripheral interface 206. Memory interface 202, processors 204, and peripherals interface 206 can be separate components or can be integrated as a part of one or more integrated circuits. The various components can be coupled by one or more communication buses or signal lines.

Processors 204 as illustrated in FIG. 2 is meant to be inclusive of data processors, image processors, central processing unit, or any variety of multi-core processing devices. Any variety of sensors, external devices, and external subsystems can be coupled to peripherals interface 206 to facilitate any number of functionalities within the architecture 200 of the exemplar mobile device. For example, motion sensor 210, light sensor 212, and proximity sensor 214 can be coupled to peripherals interface 206 to facilitate orientation, lighting, and proximity functions of the mobile device. For example, light sensor 212 could be utilized to facilitate adjusting the brightness of touch surface 246. Motion sensor 210, which could be exemplified in the context of an accelerometer or gyroscope, could be utilized to detect movement and orientation of the mobile device. Display objects or media could then be presented according to a detected orientation (e.g., portrait or landscape).

Other sensors could be coupled to peripherals interface 206, such as a temperature sensor, a biometric sensor, or other sensing device to facilitate corresponding functionalities. Location processor 215 (e.g., a global positioning transceiver) can be coupled to peripherals interface 206 to allow for generation of geo-location data thereby facilitating geo-positioning. An electronic magnetometer 216 such as an integrated circuit chip could in turn be connected to peripherals interface 206 to provide data related to the direction of true magnetic North whereby the mobile device could enjoy compass or directional functionality. Camera subsystem 220 and an optical sensor 222 such as a charged coupled device (CCD) or a complementary metal-oxide semiconductor (CMOS) optical sensor can facilitate camera functions such as recording photographs and video clips.

Communication functionality can be facilitated through one or more communication subsystems 224, which may include one or more wireless communication subsystems. Wireless communication subsystems 224 can include 802.x or Bluetooth transceivers as well as optical transceivers such as infrared. Wired communication system can include a port device such as a Universal Serial Bus (USB) port or some other wired port connection that can be used to establish a wired coupling to other computing devices such as network access devices, personal computers, printers, displays, or other processing devices capable of receiving or transmitting data. The specific design and implementation of communication subsystem 224 may depend on the communication network or medium over which the device is intended to operate. For example, a device may include wireless communication subsystem designed to operate over a global system for mobile communications (GSM) network, a GPRS network, an enhanced data GSM environment (EDGE) network, 802.x communication networks, code division multiple access (CDMA) networks, or Bluetooth networks. Communication subsystem 224 may include hosting protocols such that the device may be configured as a base station for other wireless devices. Communication subsystems can also allow the device to synchronize with a host device using one or more protocols such as TCP/IP, HTTP, or UDP.

Audio subsystem 226 can be coupled to a speaker 228 and one or more microphones 230 to facilitate voice-enabled functions. These functions might include voice recognition, voice replication, or digital recording. Audio subsystem 226 in conjunction may also encompass traditional telephony functions.

I/O subsystem 240 may include touch controller 242 and/or other input controller(s) 244. Touch controller 242 can be coupled to a touch surface 246. Touch surface 246 and touch controller 242 may detect contact and movement or break thereof using any of a number of touch sensitivity technologies, including but not limited to capacitive, resistive, infrared, or surface acoustic wave technologies. Other proximity sensor arrays or elements for determining one or more points of contact with touch surface 246 may likewise be utilized. In one implementation, touch surface 246 can display virtual or soft buttons and a virtual keyboard, which can be used as an input/output device by the user.

Other input controllers 244 can be coupled to other input/control devices 248 such as one or more buttons, rocker switches, thumb-wheels, infrared ports, USB ports, and/or a pointer device such as a stylus. The one or more buttons (not shown) can include an up/down button for volume control of speaker 228 and/or microphone 230. In some implementations, device 200 can include the functionality of an audio and/or video playback or recording device and may include a pin connector for tethering to other devices.

Memory interface 202 can be coupled to memory 250. Memory 250 can include high-speed random access memory or non-volatile memory such as magnetic disk storage devices, optical storage devices, or flash memory. Memory 250 can store operating system 252, such as Darwin, RTXC, LINUX, UNIX, OS X, ANDROID, WINDOWS, or an embedded operating system such as VxWorks. Operating system 252 may include instructions for handling basic system services and for performing hardware dependent tasks. In some implementations, operating system 252 can include a kernel.

Memory 250 may also store communication instructions 254 to facilitate communicating with other mobile computing devices or servers. Communication instructions 254 can also be used to select an operational mode or communication medium for use by the device based on a geographic location, which could be obtained by the GPS/Navigation instructions 268. Memory 250 may include graphical user interface instructions 256 to facilitate graphic user interface processing such as the generation of an interface; sensor processing instructions 258 to facilitate sensor-related processing and functions; phone instructions 260 to facilitate phone-related processes and functions; electronic messaging instructions 262 to facilitate electronic-messaging related processes and functions; web browsing instructions 264 to facilitate web browsing-related processes and functions; media processing instructions 266 to facilitate media processing-related processes and functions; GPS/Navigation instructions 268 to facilitate GPS and navigation-related processes, camera instructions 270 to facilitate camera-related processes and functions; and instructions 272 for any other application that may be operating on or in conjunction with the mobile computing device.

Memory 250 may also store other software instructions for facilitating other processes, features and applications, such as applications related to navigation, social networking, location-based services or map displays. Each of the above identified instructions and applications can correspond to a set of instructions for performing one or more functions described above. These instructions need not be implemented as separate software programs, procedures, or modules. Memory 250 can include additional or fewer instructions. Furthermore, various functions of the mobile device may be implemented in hardware and/or in software, including in one or more signal processing and/or application specific integrated circuits.

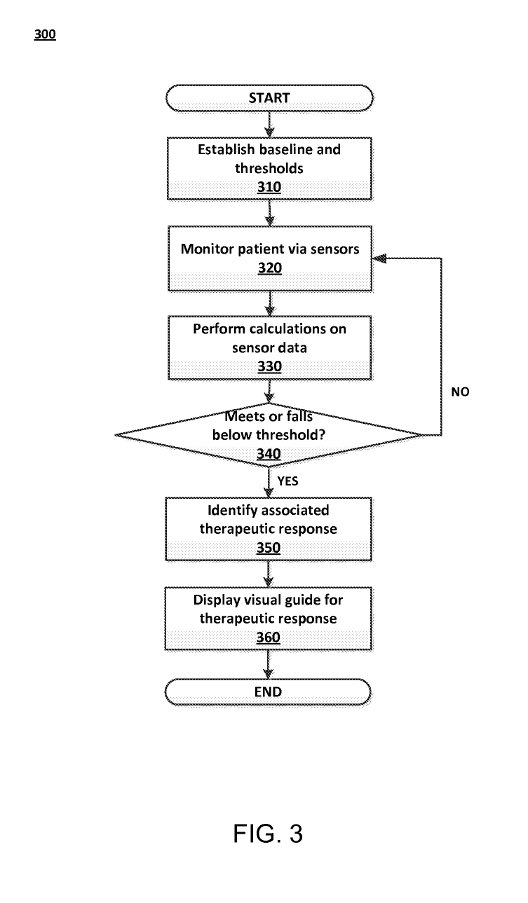

FIG. 3 is a flowchart illustrating a method for real-time vagal monitoring and intervention. The method 300 of FIG. 3 may be embodied as executable instructions in a non-transitory computer readable storage medium including but not limited to a CD, DVD, or non-volatile memory such as a hard drive. The instructions of the storage medium may be executed by a processor (or processors) to cause various hardware components of a computing device hosting or otherwise accessing the storage medium to effectuate the method. The steps identified in FIG. 3 (and the order thereof) are exemplary and may include various alternatives, equivalents, or derivations thereof including but not limited to the order of execution of the same.

In method 300 of FIG. 3, a baseline and one or more thresholds may be established for a specific person, the person may continue to be monitored via sensors, sensor data may be used in various calculations, and it may be determined whether the calculations meet a risk threshold. If so, a therapeutic intervention associated with the risk threshold may be identified, and a visual guide for the identified therapeutic intervention may be launched on a display screen of a user device.

In step 310, a baseline and one or more thresholds may be established for the person. Establishing a baseline for the person may involve using the monitor device to monitor the person for a predetermined period of time (e.g., a registration period). During the registration period, data from the sensors is gathered over time and used to identify a range of measures that are specific to the person. The range may be divided into percentiles, which may be selected to serve as a threshold.

For example, a monitor device (e.g., monitor device 110) may record electrocardiogram data, determine the heart rate and heart rate interval, and calculate a root mean square of the successive differences (RMSSD) to obtain a time-domain measure of heart rate variability. The person may exhibit a range of RMSSD measures over time. The 20^{th} percentile may serve, for example, as a threshold that may be indicative of risk, thereby triggering a therapeutic intervention. Conversely, the 40^{th} percentile may serve as a target threshold at which a triggered therapeutic intervention may cease. Such a target threshold may be within a range associated with less dangerous or risky status. While one or more thresholds may be set by default, such thresholds may also be customized by the person or an associated health care provider.

In step 320, the person may be monitored by sensors of the monitor device 110 in real-time. Monitor device 110 may be worn by the person during the course of their regular day and continue to gather data via its sensors regarding the condition of the person. Referring to the foregoing example, RMSSD measures pertaining to HRV may be updated every three seconds (or other time period as selected by the person or associated health care provider).

In step 330, calculations may be performed on the sensor data. Where the sensor data includes ECG data, for example, such calculations may be the RMSSD measures. Such calculations may take place at the monitor device 110 and sent (e.g., via a Bluetooth connection) to a synchronized user device 120. As such, monitoring data regarding the person may continuously be sent to the user device 120. The user device 120 may also perform such calculations or may perform additional calculations to obtain different metrics regarding the health of the person. The frequency of transmission between the monitor device 110 and the user device 120 may be customized as would best fit the needs of the person (e.g., to conserve battery life in circumstances when the person may not be near an outlet).

Some embodiments may involve additional calculations to evaluate and weight the effects of certain conditions. For example, it may be determined when HRV values may be impacted by mental stress by subtracting an HRV value due to exertion as determined by actigraphy data.

In some embodiments, the sensor data may be sent to a server (e.g., server 150) that is accessible to a health care provider, supervisor, or colleague associated with the person (or other designated individuals) for offline analysis. Such a server 150 may be used to store accumulated data regarding the person over time, which may be retrieved and used in various other calculations and evaluations regarding the person. Such data may also be used for quality control checks, evaluate therapeutic interventions, and to identify and eliminate artifacts. For example, premature atrial or ventricular beats can generate distorted HRV values if the premature beats are not recognized and deleted. Noise level may be identified by the monitor device 110 or the user device 120. Such information may also be used to restrict usage of sensor data (e.g., ECG segments) to that which is noise-free. Alternatively, the threshold for data use can be specified. In addition, specified parameters (e.g., 40% of previous RR duration or greater than 1.5 second pause) may be used to detect and eliminate premature or delayed beats. Off-line ECG analysis can also be used to determine the extent to which artifacts have been detected and eliminated. These same ECG data can be analyzed using other metrics of HRV as desired. Power spectral analyses can also be performed on ECG data off-line to determine sympatho-vagal balance derived from the ratio of low frequency (an index indicative of sympathetic influences) to high frequency (an index of parasympathetic influences) HRV ratio for ECG segments at least 30 seconds in duration.

In step 340, it may be determined whether a calculation performed in step 330 meets any of the risk thresholds specifically defined for the person in step 310. Referring to the example provided above, step 340 may involve determining that a received RMSSD measure meets or falls below the 20^{th} percentile of the range, thereby meeting or falling below an established risk threshold for the person. If not, normal monitoring and calculations may continue as described in steps 320 and 330. If a calculation is determined to meet or fall below a risk threshold, however, the method may proceed to step 350.

In step 350, a notification or alert is sent to the user device regarding a therapeutic intervention associated with the risk threshold identified in step 340. Such a therapeutic intervention may include deep breathing, meditation, a breathing pattern, etc. For example, a breathing pattern may consist of a proportional rhythm of 4 (inspiration)-4 (inspiratory pause)-6 (expiration)-2 (expiratory pause) at a rate of 4-6 breaths per minute. The association between the risk threshold and the associated therapeutic intervention may be set by the person and/or by an associated health care provider. Different patterns and therapeutic intervention may be associated with different thresholds. Such associations may be stored in a database on user device 120, which may also be updated and/or customized for the person. In some embodiments, various combinations of user match parameters may be specified, such that different therapeutic intervention may be selected for the person based on different circumstances.

In some instances, the therapeutic intervention may further include instructions to certain devices to implement at least some of the therapeutic intervention. For example, a therapeutic intervention may involve sending certain instructions (e.g., settings) to a ventilator that assists with breathing. Similarly, another therapeutic intervention may involve sending instructions or settings to a pacemaker. In that regard, some embodiments may further use the pacemaker or other implanted cardiac device as a monitor device 110 to gather data regarding the heart and to use such data as a parameter in its calculations or other considerations as to whether, when, and how to implement a therapeutic intervention.

In step 360, a visual guide may be displayed on a display screen of the user device 120. Such a visual guide may assist the person in implementing the therapeutic intervention identified in step 350. For example, a visual guide associated with a breathing pattern may include an inflatable ball that inflates, pauses, and deflates in accordance with the selected pattern. The visual guide may also include a vertical bar that grows and shrinks in accordance with the pattern. While the person is being guided through implementation of the therapeutic intervention, the monitor device 110 may continue to send sensor data and/or calculations in real-time. The sensor data and/or calculations may be used to determine how well the person is adhering to the selected therapeutic intervention. The display screen may be updated to provide feedback (e.g., numerical scores or percentage of fidelity to therapeutic program) so as to provide the person with information and to assist in adhering to the therapeutic intervention properly. In this regard, the type and duration of the therapeutic intervention may be adjusted as necessary in response to real-time data regarding the person.

The therapeutic intervention may last for a predetermined period of time, or when the real-time calculations are determined to meet a target threshold established for the person. In some instances, the target threshold must be determined to be met for a predetermined period of time before the therapeutic intervention ceases. Once the target threshold has been reached (or the time period has elapsed), the cool-down cycle may be launched, which may likewise be associated with a visual guide.

In some instances, the user device 110 may allow the user to select and launch a therapeutic intervention upon request, based on a designated schedule, etc. For example, the user and/or their health professional may schedule certain therapeutic exercises, as well as designate the frequency and duration. Alternatively, the user may launch a therapeutic intervention as needed or desired.

In addition, user device 110 may provide a personalized dashboard with real-time data, historical data, and/or detected trends in data pertaining to the user (e.g., user profile). An exemplary dashboard may include, for example, RR internal, heart rate, RMSSD, thresholds (e.g., percentile milestones), respiration rate, posture, frequency and duration of therapeutic interventions and their effects on the other biodata, fidelity to the therapeutic invention, calculations regarding mental stress, accuracy as verified by offline analysis, etc. In some instances, data may be continuously retrieved from the server for display in the dashboard. Such data may also be communicated between various servers and devices accessible to designated medical or other supervisory personnel.

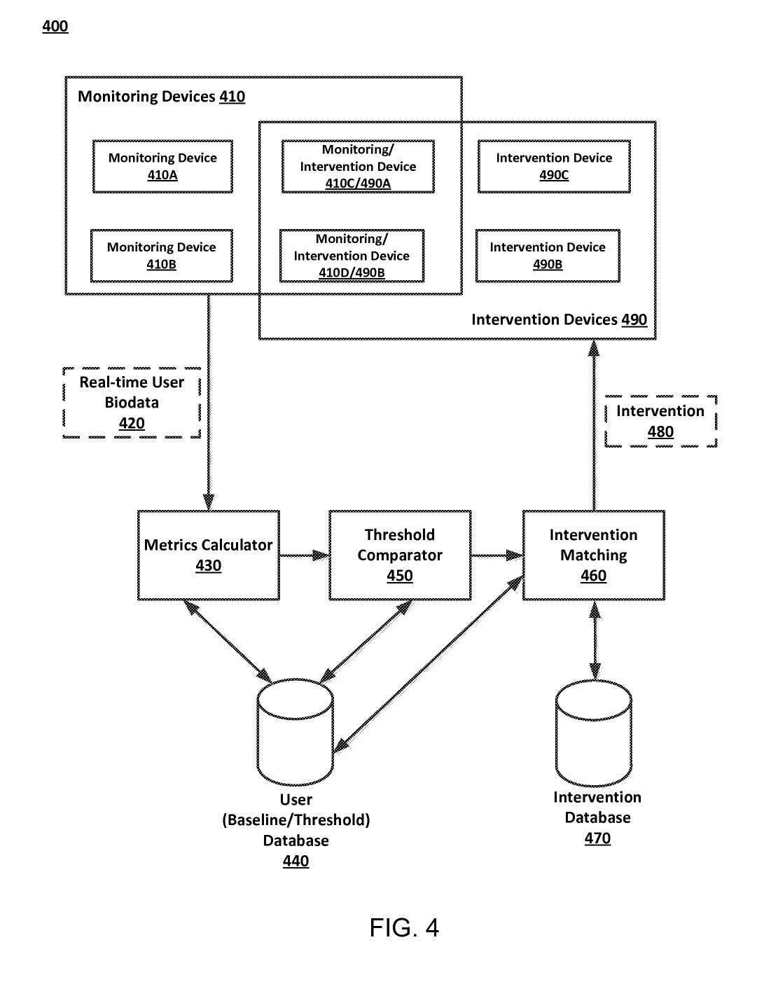

FIG. 4 is diagram of an exemplary system 400 for real-time vagal monitoring and intervention. System 400 may include monitoring devices 410 (410A-410D), which monitor the user and send real-time biodata 420A to metrics calculator 430. Metrics calculator 430 is further in communication with user database 440. Using the real-time biodata 420 received from monitoring device(s) 410 and/or information provided by user database 440, metric calculator 430 generates certain metrics. For example, using data regarding heart electrical activity detected by ECG electrodes in one or more of the monitoring devices 410, the metrics calculator 430 may determine HRV measures. Such HRV measures may be sent to user database 440 for short-term or long-term storage, as well as sent to threshold comparator 450 for comparison to thresholds established for the user.

User database 440 may additionally receive and store user input, user biodata, and various metrics calculated by metric calculator 430 based on such biodata. Such metrics may include a baseline HRV for the user, as well as various risk and target thresholds established for the user. Such baselines and thresholds may be calculated as described above, as well as sent to threshold comparator 450 for comparison to current HRV measures.

When threshold comparator 450 detects a match between a current HRV measure and a risk threshold, the intervention matching module 460 may be triggered to identify which intervention (e.g., from the inventions stored in intervention database 470) to launch. The selection or identification of the intervention may be based on the risk threshold, as well as various other factors stored for the user or that may be designated by the user or by an associated health care provider or administrator.

Information and instructions regarding the selected intervention 480 may be sent to one or more intervention devices 490D. As illustrated, some intervention devices may also be monitoring devices. As such, a single device (e.g., monitoring/intervention devices 410C/490A and 410C/490B) may be capable of both monitoring and launching an intervention. Alternatively, the functions of monitoring and launching the intervention may be performed by different devices.

The interventions database 470 may be updated to add new interventions, remove outdated interventions, update interventions, associate interventions with different thresholds and parameters, etc. Such updates may be provided from the user, an associated health care provider, or other designated individual.

The present invention may be implemented in an application that may be operable using a variety of devices. Non-transitory computer-readable storage media refer to any medium or media that participate in providing instructions to a central processing unit (CPU) for execution. Such media can take many forms, including, but not limited to, non-volatile and volatile media such as optical or magnetic disks and dynamic memory, respectively. Common forms of non-transitory computer-readable media include, for example, a floppy disk, a flexible disk, a hard disk, magnetic tape, any other magnetic medium, a CD-ROM disk, digital video disk (DVD), any other optical medium, RAM, PROM, EPROM, a FLASHEPROM, and any other memory chip or cartridge.

Various forms of transmission media may be involved in carrying one or more sequences of one or more instructions to a CPU for execution. A bus carries the data to system RAM, from which a CPU retrieves and executes the instructions. The instructions received by system RAM can optionally be stored on a fixed disk either before or after execution by a CPU. Various forms of storage may likewise be implemented as well as the necessary network interfaces and network topologies to implement the same.

While various embodiments have been described above, it should be understood that they have been presented by way of example only, and not limitation. The descriptions are not intended to limit the scope of the invention to the particular forms set forth herein. Thus, the breadth and scope of a preferred embodiment should not be limited by any of the above-described exemplary embodiments. It should be understood that the above description is illustrative and not restrictive. To the contrary, the present descriptions are intended to cover such alternatives, modifications, and equivalents as may be included within the spirit and scope of the invention as defined by the appended claims and otherwise appreciated by one of ordinary skill in the art. The scope of the invention should, therefore, be determined not with reference to the above description, but instead should be determined with reference to the appended claims along with their full scope of equivalents.

1. (canceled)

2. The monitoring device of claim 1, further comprising a display screen that provides the visual guide regarding the identified therapeutic intervention, and wherein the display screen provides the visual guide until the user wireless interface receives one or more subsequent HRV measures from the monitor device that meet a HRV target threshold.

3. The monitoring device of claim 2, wherein the user device further comprises memory that stores information regarding the person, and wherein the stored information includes multiple HRV thresholds specific to the person.

4. The monitoring device of claim 3, wherein the user device further comprises a user device processor, wherein the user device processor calculates multiple HRV thresholds during a predefined registration period, and wherein the HRV thresholds include the HRV risk threshold and the HRV target threshold.

5. The monitoring device of claim 4, wherein the user device processor calculates each threshold by monitoring the person during the predefined registration period and establishing a baseline, wherein each HRV threshold is calculated based on the established baseline.

6. The monitoring device of claim 5, wherein monitoring the person comprises instructing the monitor device to calculate and transmit multiple HRV measures during the predefined registration period.

7. The non-transitory computer-readable storage medium of claim 27, wherein the user device comprises a user device processor that calculates multiple HRV thresholds during a predefined registration period, and wherein the HRV thresholds include the HRV risk threshold and the HRV target threshold, and wherein the calculation of each threshold by the user device processor further comprises determining a distribution of the HRV measures calculated during the predefined registration period and breaking the determined distribution into percentiles.

8. The non-transitory computer-readable storage medium of claim 7, wherein the multiple HRV thresholds specific to the person includes at least one HRV threshold set by the person or by a health care provider of the person.

9. The non-transitory computer-readable storage medium of claim 27, wherein the user device comprises user device memory that stores multiple therapeutic interventions, each associated with a different HRV risk threshold.

10. The system of claim 9, wherein the association between at least one of the HRV risk thresholds and the associated therapeutic intervention is designated by the person or by a health care provider of the person.

11. The system of claim 9, wherein the user device memory further stores a schedule specific to the person, and wherein the visual guide comprises a display screen that generates a notification regarding one or more therapeutic interventions in accordance with the schedule.

12. The system of claim 9, wherein the user device further comprises a user interface that receives a selection of one of the stored therapeutic interventions, and wherein the user device display provides a visual guide associated with the selected therapeutic intervention.

13-20. (canceled)

21. A monitoring device for real-time vagal monitoring and intervention, the monitoring device comprising:
one or more sensors that detects electrical activity in a heart of the person;
a processor that executes instructions stored in memory, wherein execution of the instructions by the processor calculates multiple measures regarding heart rate variability (HRV) based on electrical activity detected by the sensors; and
a wireless interface that continuously transmits each calculated HRV measure over a wireless network to a user device that:
determines when the received HRV measure meets a predetermined HRV risk threshold specific to the person,
identities a therapeutic intervention associated with the HRV risk threshold, and
provides a visual guide regarding the identified therapeutic intervention.

22-26. (canceled)

27. A non-transitory computer-readable storage medium, having embodied thereon a program executable by a processor to perform a method for real-time vagal monitoring and intervention, the method comprising:
detecting electrical activity in a heart of the person;
calculating one or more measures regarding heart rate variability (HRV) based on the detected electrical activity; and
continuously transmitting each calculated HRV measure over a wireless network to a user device that:
determines when the received HRV measure meets a predetermined HRV risk threshold specific to the person,
identities a therapeutic intervention associated with the HRV risk threshold, and
provides a visual guide regarding the identified therapeutic intervention.

28. A non-transitory computer-readable storage medium, having embodied thereon a program executable by a processor to perform a method for real-time vagal monitoring and intervention, the method comprising:
receiving multiple continuous heart rate variability (HRV) measures sent over a wireless communication network from a monitor device, each calculated HRV measure regarding electrical activity of a heart of a person;
determining when the received HRV measure meets a predetermined HRV risk threshold specific to the person;
identifying a therapeutic intervention associated with the HRV risk threshold; and
providing a visual guide regarding the identified therapeutic intervention.

29-32. (canceled)
